(19) United States
(12) Patent Application Publication
(10) Pub. No.: US 2024/0215813 A1
(43) Pub. Date: Jul. 4, 2024

(54) Self-Cleaning Dental Mirror

(71) Applicant: Meilin Howard, Aledo, TX (US)

(72) Inventor: Meilin Howard, Aledo, TX (US)

(21) Appl. No.: 18/401,583

(22) Filed: Dec. 31, 2023

Related U.S. Application Data
(60) Provisional application No. 63/478,188, filed on Jan. 2, 2023.

(51) Int. Cl. A61B 1/253; A61B 1/00; A61B 1/12

(52) U.S. Cl. CPC A61B 1/253; A61B 1/00032; A61B 1/00042; A61B 1/126

(57) ABSTRACT

A self-cleaning dental mirror which allows for procedures to continue uninterrupted by using a wiper to clear away debris. The self-cleaning dental mirror is activated through a battery system which activates the wiper system from a button press, sensor system, or internal timer which pushes the wiper along the surface of the mirror and scraping the debris from the wiper when returning.

### CROSS-REFERENCE TO RELATED APPLICATIONS

This application claims benefit to provisional application U.S. 63/478,188 filed on Jan. 2, 2023, and is incorporated in its entirety.

### PRIOR ART

While prior art such as U.S. Pat. No. 7,455,519 exist, it uses the mechanical force of the user to remove the debris from the dental mirror. The prior art also doesn't have a sensor which allows for the automatic removal of debris from the dental mirror.

### BACKGROUND OF THE INVENTION

Most dental mirrors require manual cleaning. This normally results in the dentist taking a moment to stop the procedure and rinsing off the dental mirror and then resuming the procedure. There are different types of solutions or wipes the can be used to make the mirror somewhat waterproof, but nothing effectively addresses solid debris.

Dental assistants can help rinse and dry the mirror with air-water syringe, but the physical power generated from the air-water syringe is often not enough to dislodge solid debris from a drilling procedure.

An operator usually has to be wiped the mirror every 5-10 seconds if the mirror has to be placed close to the tooth being worked on. Stopping the drilling every 5-10 slows down the procedure and extends discomfort for the patient.

### SUMMARY OF THE INVENTION

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements and are not drawn to scale. least one specification heading is required.

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention.

The present subject matter is a lithium battery powered dental mirror with a wiper design. The self-cleaning dental mirror is activated by a button on the handle or using a sensor similar to that of a rain sensor. A plastic wiper with silicone coating is used to clean off solid debris that's generated during dental procedures. It prevents the need for the operator to pause the procedure and manually clean off mirror with gauze or wipe outside of the patient's mouth.

In some embodiments, the wiper can be set to an automatic timer which wipes the mirror clean at various intervals. These intervals are typically between five and ten seconds.

In some embodiments, a sensor is embedded into the dental mirror and whenever the sensor detects it is covered by debris, the wiper is activated.

In some embodiments, the wiper pushes the debris from the base of the mirror towards the furthest outer edge of the mirror. In some embodiments the wiper pushes the debris from left to right or from right to left.

In some embodiments, an internal water source is used in combination with the wiper system to clean off the mirror.

In some embodiments, the lithium battery powers a heating element to prevent fogging on the mirror.

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

### BRIEF DESCRIPTION OF DRAWINGS

FIG. 1 shows a perspective view of the self-cleaning dental mirror with debris still on the mirror.

FIG. 2 shows a perspective view of the self-cleaning dental mirror while the wiper is activated.

FIG. 3 shows a perspective view of the self-cleaning dental mirror after the wiper has removed the debris.

FIG. 4 shows a perspective view of the self-cleaning dental mirror after the wiper has reset to its original position.

FIG. 5 shows a perspective view of the self-cleaning dental mirror after the wiper has reset to its original position and the activation button is exposed.

FIG. 6 shows a perspective view of the self-cleaning dental mirror while the wiper is activated.

### DETAILED DESCRIPTION OF THE INVENTION

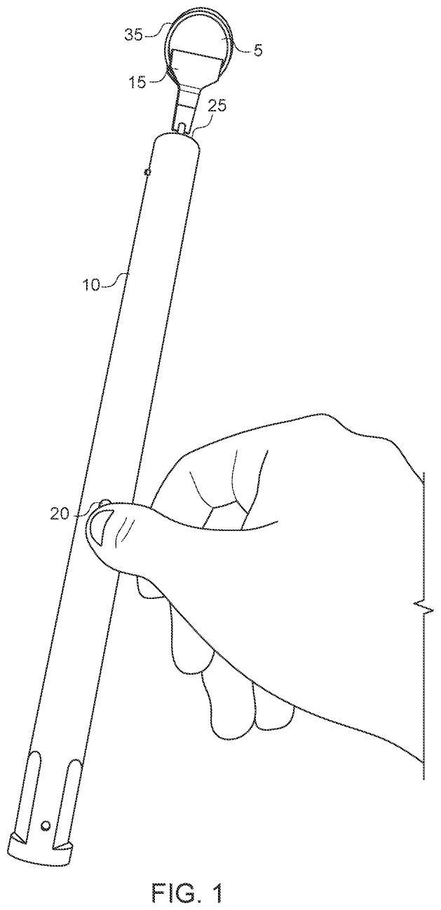

FIG. 1 shows a perspective view of the self-cleaning dental mirror wherein mirror 5 is affixed to the handle 10 of the self-cleaning dental mirror. Wiper 15 is made of plastic or other suitable material and may be coated with silicone to closely push along the surface of the mirror when removing debris. Activation button 20 is located on the front of handle 10 near the natural resting place of the dentist's thumb.

In some embodiments, a single press of activation button 20 begins a continuous wipe rotation that lasts five seconds. In other embodiments, the dentist is able to activate individual mirror wipes as needed. In further embodiments, sensor 25 is located on the interior of handle 10 wherein the sensor is able to detect when debris is on the mirror and activates without input from the dentist.

Heating element 35 is located in the interior of mirror 5 which helps prevent fogging.

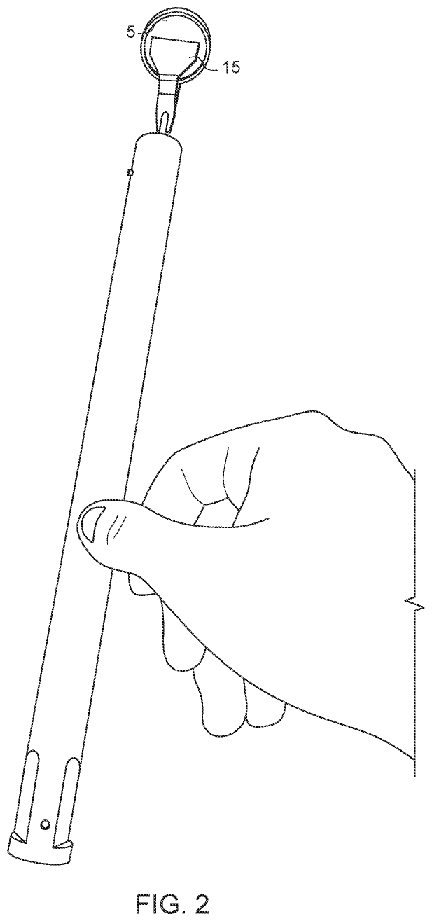

FIG. 2 shows a perspective view of the self-cleaning dental mirror while the wiper is activated wherein wiper 15 pushes along the surface of mirror 5 in a manner to remove debris off the opposite side of the mirror.

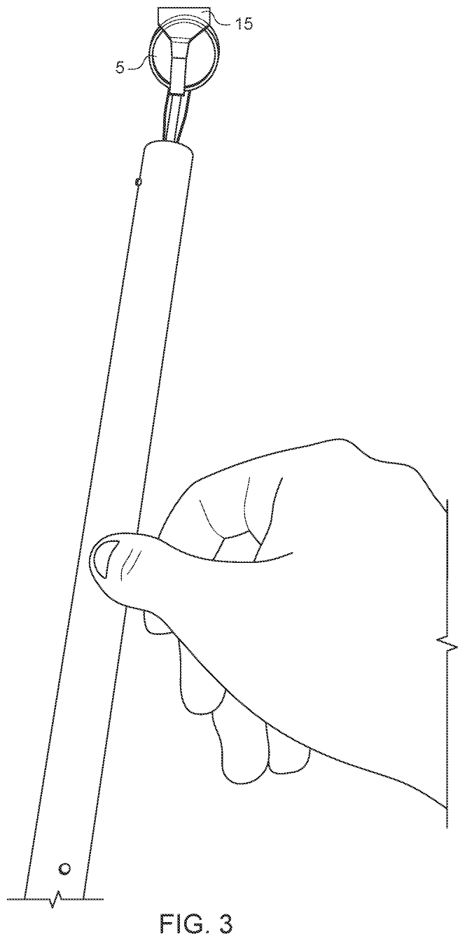

FIG. 3 shows a perspective view of the self-cleaning dental mirror after the wiper has removed the debris wherein the debris has been pushed off the edge of mirror 5 in a manner that allows the debris to be scraped from wiper 15 and force the debris to the backside of mirror 5 or drop off from the mirror entirely.

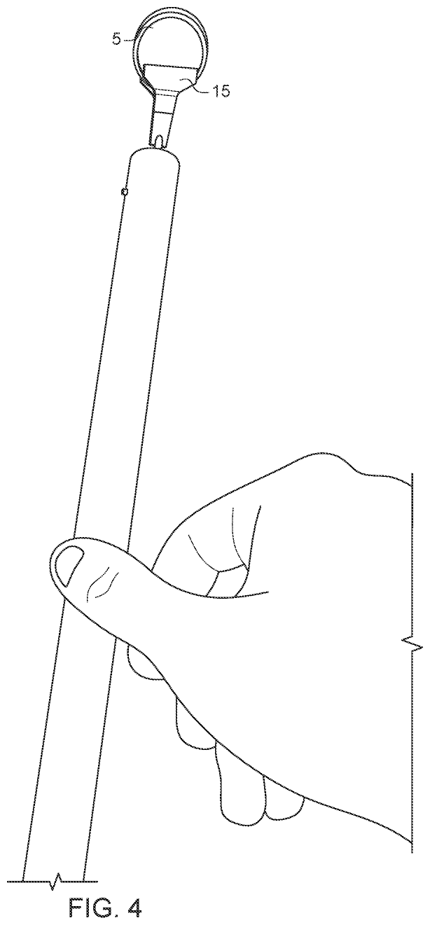

FIG. 4 shows a perspective view of the self-cleaning dental mirror after the wiper has reset to its original position wherein wiper 15 has been scraped clean and is able to provide a second wipe on the way back to its starting position.

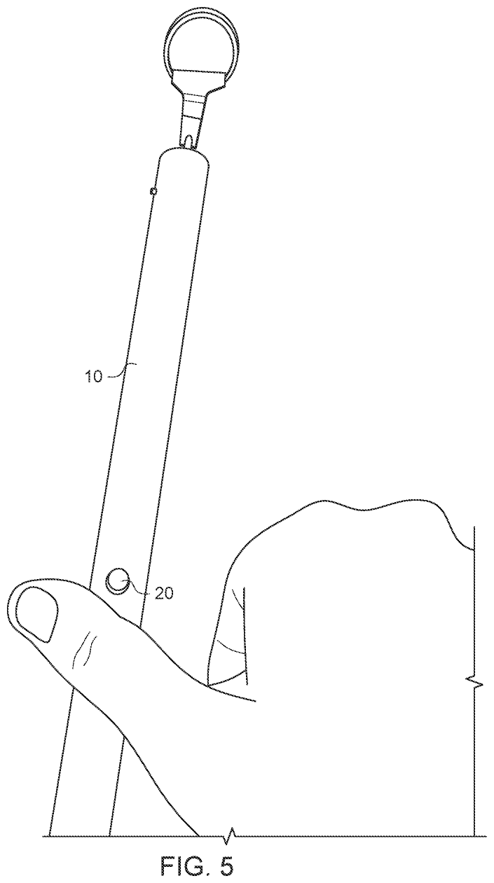

FIG. 5 shows a perspective view of the self-cleaning dental mirror after the wiper has reset to its original position and the activation button is exposed wherein activation button 20 is located on the front of handle 10 near the natural resting place of the dentist's thumb and activation button 20 may be used to activate a variety of wiper settings.

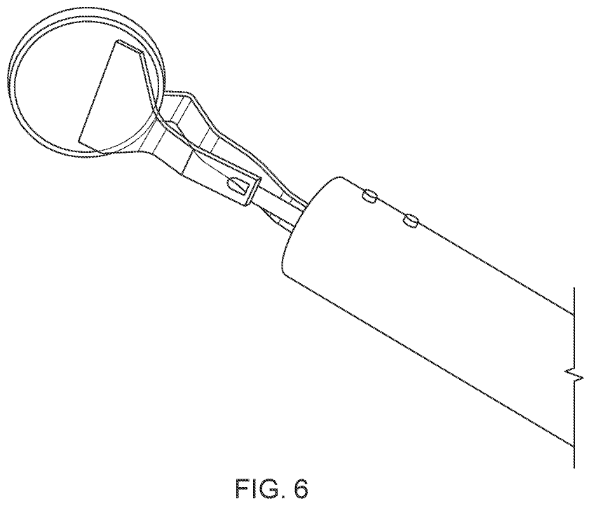

FIG. 6 shows a perspective view of the self-cleaning dental mirror while the wiper is activated at an angle that would be typical for dentist if it were being actively used on a patient.

1. A self-cleaning dental mirror comprising of a handle, mirror, lithium-ion battery, sensor, activation button, timer, heating element, and wiper.

2. The self-cleaning dental mirror as in claim 1, wherein the wiper and other features are powered by the lithium-ion battery.

3. The self-cleaning dental mirror as in claim 1, wherein the wiper is operated by the activation button.

4. The self-cleaning dental mirror as in claim 1, wherein the wiper is controlled by a sensor.

5. The self-cleaning dental mirror as in claim 4, wherein the sensor detects when debris is on the mirror and activates the wiper.

6. The self-cleaning dental mirror as in claim 1, wherein the wiper is controlled by a timer.

7. The self-cleaning dental mirror as in claim 6, wherein the timer may be set to various intervals.

8. A self-cleaning dental mirror comprising of a handle, mirror, lithium-ion battery, sensor, buttons, timer, wiper, heating element, and water system.

9. The self-cleaning dental mirror as in claim 8, wherein the water system is controlled by a button, sensor, or timer.

10. The self-cleaning dental mirror as in claim 8, wherein the water system assists the wiper with cleaning the mirror.
